(19) United States
(12) Patent Application Publication
(10) Pub. No.: US 2016/0220186 A9
(43) Pub. Date: Aug. 4, 2016

(54) Detachable Wireless Motion System for Human Kinematic Analysis

(71) Applicants: Timothy S. Clark, Moss Beach, CA (US); John C. Litschert, Fort Collins, CO (US)

(72) Inventors: Timothy S. Clark, Moss Beach, CA (US); John C. Litschert, Fort Collins, CO (US)

(21) Appl. No.: 14/509,832

(22) Filed: Oct. 8, 2014

Related U.S. Application Data
(60) Provisional application No. 61/890,299, filed on Oct. 13, 2013.

(51) Int. Cl. A61B 5/00; A61B 5/11

(52) U.S. Cl. CPC A61B 5/6807; A61B 5/112; A61B 5/0004; A61B 5/7278; A61B 5/1122; A61B 5/7246; A61B 2560/0475; A61B 2503/10

(57) ABSTRACT

A system for determining user kinematic characteristics includes a detachable motion sensor, a processing system, and a wireless transceiver. The motion sensor may be coupled with a user's footwear in order to generate one or more signals corresponding to the motion of the user's foot/feet. The processing system is in communication with the motion sensor and is programmed to use the one or more signals to determine one or more kinematic parameters. The present invention measures various parameters about each individual stride. The stride based kinematic characteristics may include, but are not limited to, pitch, roll, yaw, vertical position, horizontal position, horizontal velocity, vertical velocity, distance traveled, foot strike, foot strike classification, toe off, contact time, stride rate, stride length, rate of pronation, maximum pronation, rate of plantarflexion and dorsiflexion, swing velocity, and pitch-roll signature.

This application claims benefit of Provisional Application 61/890,299 filed Oct. 13, 2013 entitled “Detachable Wireless Motion System for Human Kinematic Analysis”.

### BACKGROUND

Monitoring of an athlete's kinematics both in training and in competition is important in the development and implementation of new approaches towards performance improvement as well as injury analysis and prevention.

Motion sensing devices are frequently used in order to determine the motion of an athlete. For example, such devices may sense motion parameters such as acceleration, angular rates, velocity, stride distance, total distance, speed, stride rate, and the like, for use in the training and evaluation of athletes, and the rehabilitation of the injured.

There are a number of solutions that measure kinematic parameters in one plane (X/Y) and the orientation (pitch) of an athlete's foot. These systems provide valuable insight into the biomechanics of motion, but fail to resolve the full 6D movement of the athlete's foot. 6D—in the context of stride based kinematics—representing both the position (X, Y, Z) as well as the orientation (pitch, roll, yaw) of the athlete's foot.

These designs, having focused on XY-plane stride kinematics, are implemented as single foot solutions. This assumes left/right symmetry, which for some metrics is safe, but for many, is an invalid assumption. Metrics like stride rate, velocity, even contact time (to some degree) will tend to be highly symmetric. However, pronation velocity, pronation angle, even pitch at footstrike, among others, can be radically different between an athlete's right and left sides. The disclosed detachable measurement system may be optionally implemented as either single (right or left) or both feet—providing full 6D space/orientation kinematic parameters in each combination.

### BRIEF SUMMARY OF THE INVENTION

Embodiments of the present invention provide a system for determining athletic kinematic characteristics. The system includes an inertial sensor, a processing system, and a wireless transceiver. The inertial sensor may be coupled with a user's footwear in order to generate one or more signals corresponding to the motion of the user's foot/feet. The processing system is in communication with the inertial sensor and is programmed to use the one or more signals to determine one or more kinematic characteristics. The present invention measures various parameters about each individual stride rather than assuming a given fixed rate. The stride based kinematic characteristics may include, but are not limited to, pitch, roll, yaw, vertical position, horizontal position, horizontal velocity, vertical velocity, distance traveled, foot strike, foot strike classification, toe off, contact time, stride rate, stride length, rate of pronation, maximum pronation, rate of plantarflexion and dorsiflexion, swing velocity, and pitch-roll signature.

It is to be understood that both the foregoing general description and the following detailed description are exemplary and explanatory only and are not necessarily restrictive of the invention claimed. The accompanying drawings, which are incorporated in and constitute a part of the specification, illustrate embodiments of the invention and together with the general description, serve to explain the principles of the invention.

### BRIEF DESCRIPTION OF THE DRAWINGS

FIG. 1 depicts a side view of the disclosed motion sensing system affixed to the rear of a shoe, and relevant axes.

FIG. 2 shows the motion sensing system, this time looking at the rear of the shoe, again showing the relevant axes.

FIG. 3 shows the various cycles of foot movement during walking or running with the corresponding pitch and roll data used to determine various kinematic parameters (including Foot Strike, Pronation, Toe Off, and Swing).

FIG. 4 shows the Pitch orientation component of the device relative to the World Coordinate System (Ground).

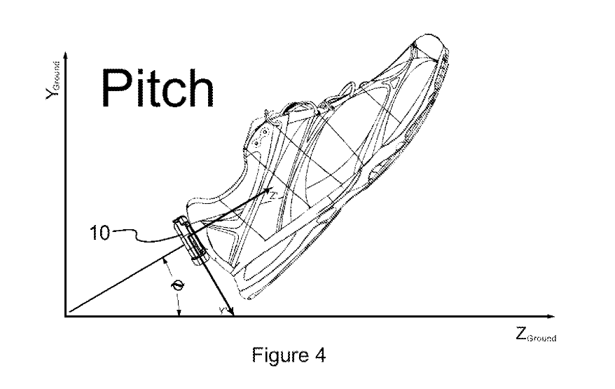

FIG. 5 shows the Roll orientation component of the device relative to the World Coordinate System (Ground).

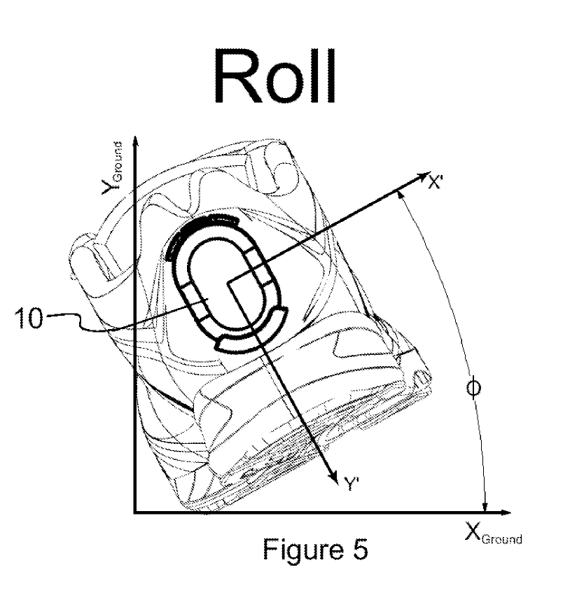

FIG. 6 shows the Yaw orientation component of the device relative to the World Coordinate System (Magnetic North).

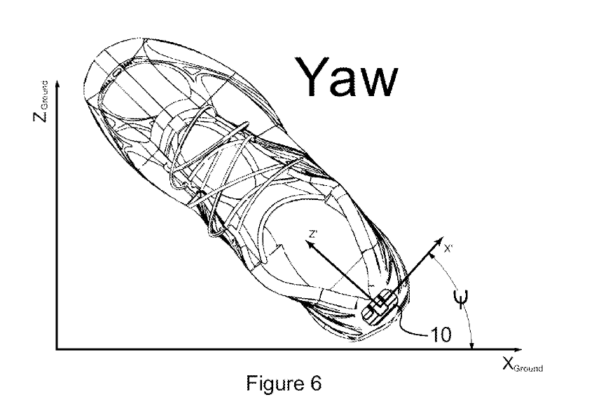

FIG. 7 is a block diagram of the motion sensing system.

FIG. 8 depicts the flow of information within the Motion Processing Unit.

FIG. 9 highlights the calculations performed within the Digital Motion Processor in order to determine the Corrected Quaternion (orientation) components.

FIG. 10 depicts a data flow diagram within the Application Processor used to calculate the Stride Based Metrics.

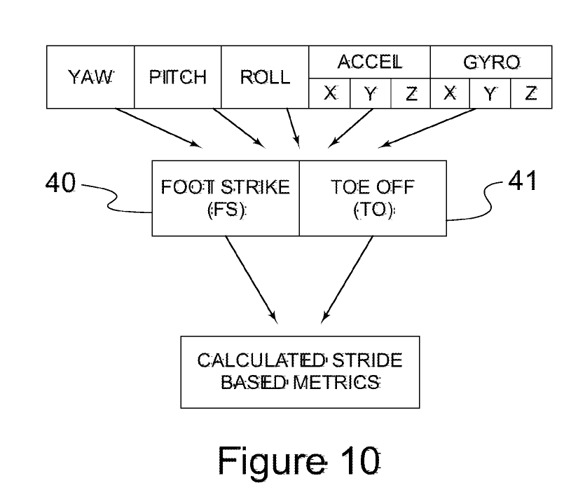

FIG. 11 shows the rotations used in the Euler 3,2,1 sequence to convert the Corrected Quaternion to Pitch, Roll, and Yaw.

FIG. 12 depicts the compensated accelerometer and gyroscope data, along with computed pitch, roll, and yaw—which are used in subsequent calculations below to determine various kinematic metrics.

FIG. 13 is an example visualization of the stride based metrics, in this case showing histograms of various parameters over the course of a typical run.

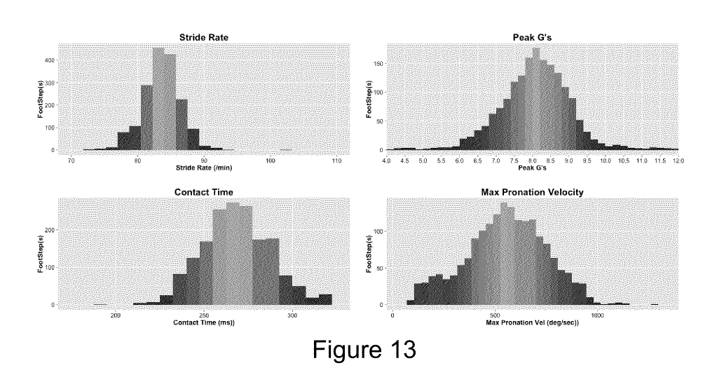

FIG. 14 contains 2D density plots of kinematic parameters, this time showing the relationship between two metrics (Contact Time vs. Stride Rate, and Peak G's vs. Stride Rate).

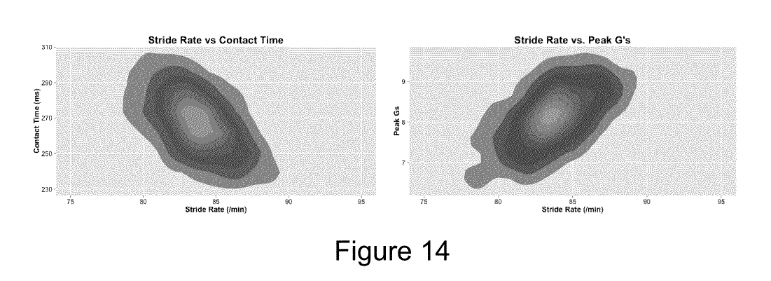

FIG. 15 is an angle-angle 2D density plot of Pitch vs. Roll for the duration of the run, highlighting areas where pitch and roll values are most frequently encountered. With Foot Strike, Max Pronation, Toe Off, Pitch Min, and Pitch Max densities overlayed.

FIG. 16 is an example of a runScore polar area chart, showing the relative differences between a plurality of metrics.

FIG. 17 shows a possible way that different footwear could be compared using any number of kinematic metrics.

FIG. 18 is an example of how a pair of shoes might be monitored over time to see how individual kinematic metrics change over the life of the shoe.

FIG. 19 depicts the use of aggregate data from a number of runners at a specific event, showing both mean and variance of kinematic metrics over the course of the event.

FIG. 20 shows how the kinematic data can be used to visualize the footstrike pattern for a given user on a given pair of shoes.

### DETAILED DESCRIPTION AND BEST MODE OF IMPLEMENTATION

The following detailed description of embodiments of the invention references the accompanying drawings. The embodiments are intended to describe aspects of the invention in sufficient detail to enable those skilled in the art to practice the invention. Other embodiments can be utilized and changes can be made without departing from the scope of the claims. The following detailed description is, therefore, not to be taken in a limiting sense. The scope of the present invention is defined only by the appended claims, along with the full scope of equivalents to which such claims are entitled.

In this description, references to “one embodiment”, “an embodiment”, or “embodiments” mean that the feature or features being referred to are included in at least one embodiment of the technology. Separate references to “one embodiment”, “an embodiment”, or “embodiments” in this description do not necessarily refer to the same embodiment and are also not mutually exclusive unless so stated and/or except as will be readily apparent to those skilled in the art from the description. For example, a feature, structure, method, etc. described in one embodiment may also be included in other embodiments, but is not necessarily included. Thus, the present technology can include a variety of combinations and/or integrations of the embodiments described herein.

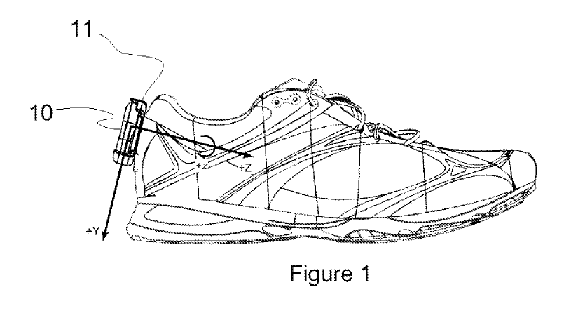

FIGS. 1 & 2 show the disclosed detachable motion sensing system 10 preferably attached to the rear portion of a shoe using mount 11.

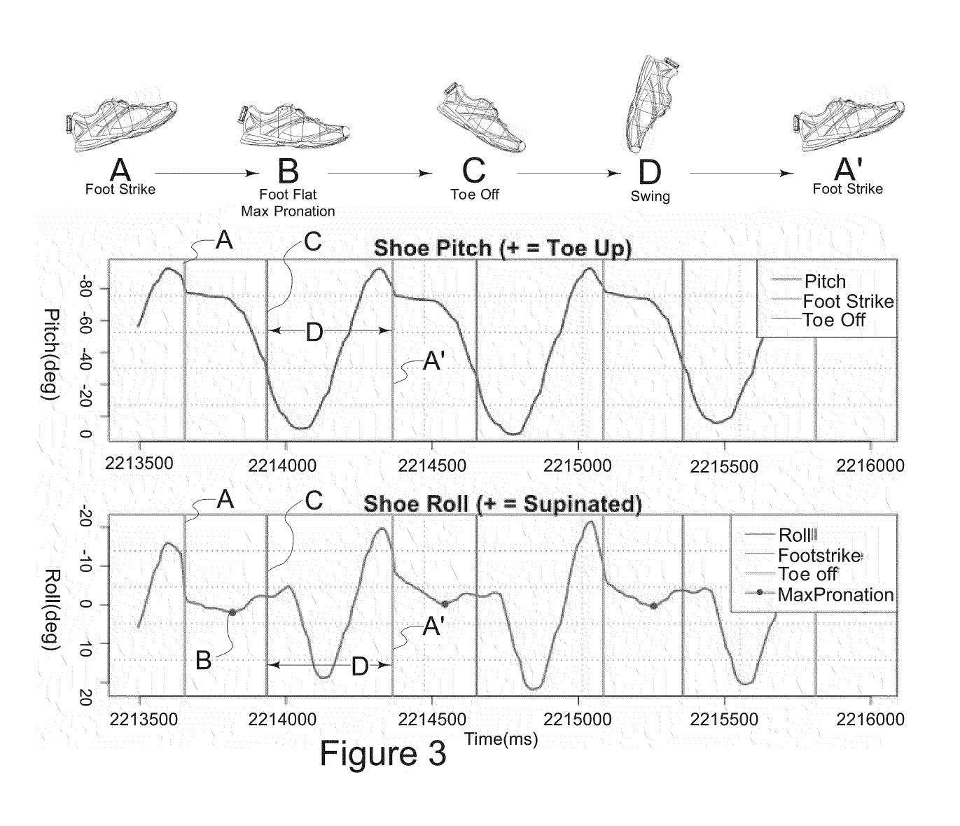

FIG. 3 shows various stages of stride in a runner (3 complete gait cycles are shown). The foot strikes the ground as indicated at locations A, A′, then continues through the pronation phase, indicated at B, then begins to pitch down as indicated at C in FIG. 3 as the toe prepares to take off. The swing phase indicated at D follows as the leg passes through the air. Following this, the foot pitches up as it prepares to strike the ground as indicated at A′ and then repeats the cycle. These linear accelerations, decelerations, rates of rotation, and changes in orientation are utilized in the present invention to determine stride kinematics as described below.

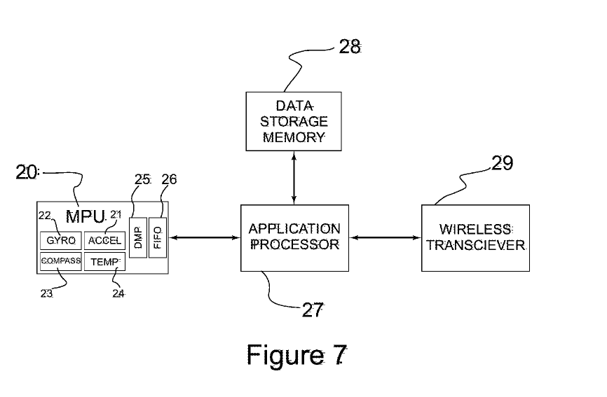

The information to permit stride based kinematic analysis is obtained via a suitable Motion Processing Unit (MPU) 20—comprised of sensors, preferably a 3D Accelerometer 21, a 3D Gyroscope 22, and (optionally) a 3D Compass 23 as shown in FIG. 7. These sensors are in communication with a suitable Digital Motion Processor (DMP) 25 that performs high precision calculations at the higher sensor sampling rates, storing the results in FIFO memory 26 for retrieval by a suitable Application Processor 27. It is worth noting that the Motion Processing Unit 20 can be implemented as a single packaged solution, in order to minimize axial misalignment errors between the various sensors, but that it may also be constructed from physically separate sensing and processing elements.

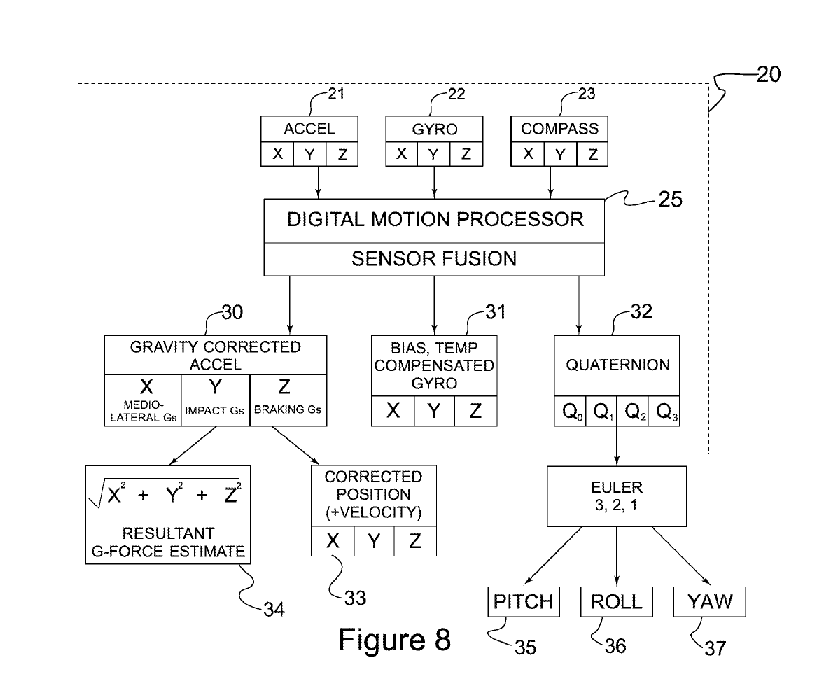

As shown in FIG. 7, the Application Processor 27 is in communication with the Motion Processing Unit 20, in order to receive the data calculated as in FIG. 8—including, but not limited to, Gravity Corrected Accelerations (X,Y,Z) 30, Bias and Temperature Compensated Angular Rates (X,Y,Z) 31, and Corrected Quaternion (Q0,Q1,Q2,Q3) 32. The Application Processor 27 further using data 30 in order to compute Position (X,Y,Z) 33, a G-Force Estimate (ImpactGs, BrakingGs, Medial-Lateral Gs) 34, and Euler Angles (Pitch, Roll, Yaw) 35, 36, 37.

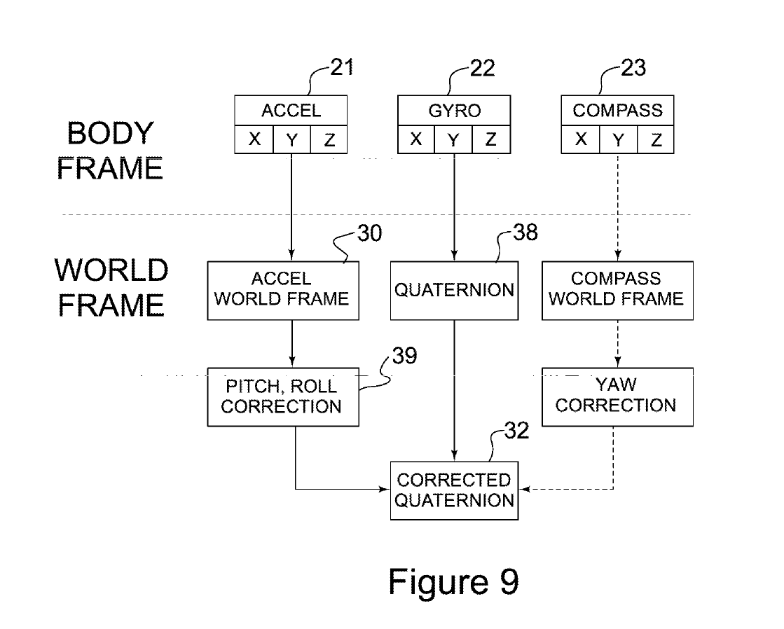

The Corrected Quaternion 32 shown in FIG. 9 is computed via a technique referred to as Sensor Fusion. Sensor fusion describes the method to derive a single, high accuracy estimate of device orientation and/or position, combining the output of various sensors. While there are many techniques to perform sensor fusion, this section will describe the basic steps required for a simple form of sensor fusion. The goal is to calculate a device Quaternion from which the orientation, gravity, rotation vector, rotation matrix, and Euler angles can be derived.

Step 1: Convert Gyroscope 22 angular rate to a quaternion representation 38, where w(t) is the angular rate and q(t) is the normalized quaternion.

dq(t)/dt=½w(t)*q(t)

Step 2: Convert Accelerometer data to world coordinates. This means using the Quaternion above to get the appropriate coordinate system in world-frame motion. Here A_{b}(t) is in the body coordinates of the device 1, while A_{w}(t) is in world-frame.

A_{w}(t)=q(t)*A_{b}(t)*q(t)′

Step 3: Create an acceleration measurement feedback quaternion 39 as below.

qf(t)=[0A_{wy}(t)−A_{wx}(t)0]*gain

Step 4: Once converted to world coordinates, accelometer feedback and gain is used to generate a feedback quaternion which is then added to the previous quaternion along with the gyro generated quaternion. The result is a Corrected Quaternion 32 that will track the gyroscope measured data, but will drift towards the accelerometer measurement, according to the value chosen for gain. Similarly, compass data can be added to the yaw component of the quaternion in order to correct for drift in yaw.

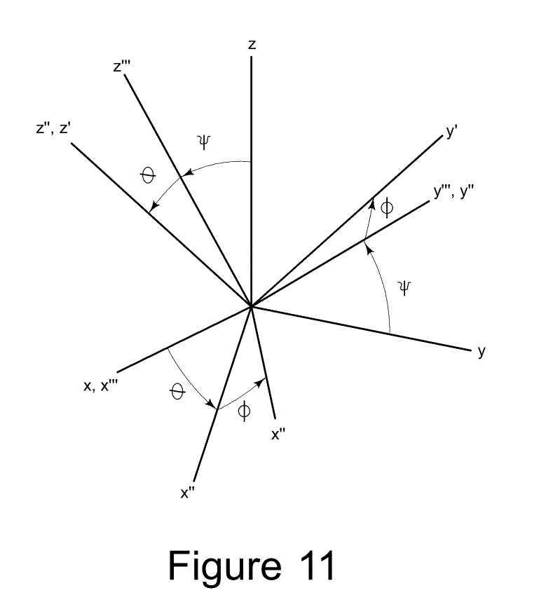

Shown in FIG. 8, the device orientation Pitch 35, Roll 36, and Yaw 37 can be computed from the Corrected Quaternion 32 via a series of matrix rotations (depicted in FIG. 11) as described by Euler's Theorem.

We associate a quaternion with a rotation around an axis by the expressions:

q₀=cos(α/2)

q₁=sin(α/2)cos(βx)

q₂=sin(α/2)cos(βy)

q₃=sin(α/2)cos(βz)

where α is a simple rotation angle (the value in radians of the angle of rotation) and cos(βₓ), cos(β_{y}) and cos(β_{z}) are the “direction cosines” locating the axis of rotation (Euler's Theorem). From this we can derive the following rotation matrix:

q₀²+q₁²−q₂²−q₃² 2(q₁q₂−q₀q₃) 2(q₀q₂−q₁q₃) 2(q₁q₂+q₀q₃) q₀²−q₁²+q₂²−q₃²2(q₂q₃−q₀q₁) 2(q₁q₃−q₀q₂) 2(q₀q₁+q₂q₃) q₀²−q₁²−q₂²+q₃²

Pitch 35, Roll 36, and Yaw 37 can thus be computed by the following equations.

Θ=a tan 2(2(q0q1+q2q3), 1−2(q1²+q2²))

Φ=arcsin(2(q₀q₂−q₃q₁))

Ψ=a tan 2(2(q0q3+q1q2), 1−2(q2²+q3²))

Having determined device orientation, it is now possible to determine device Position and Velocity (X,Y,Z) 33 by integrating the Gravity Corrected Accelerations (X,Y,Z) 30 as follows:

$${A}_{x \text{-} body} ( t ) = {A}_{x} ( t ) - g * sin ( Φ x ( t ) )$$

$${A}_{y \text{-} body} ( t ) = {A}_{y} ( t ) - g * sin ( Φ y ( t ) )$$

$$\frac{{A}_{x \text{-} world} ( t ) = {A}_{x \text{-} body} ( t ) * sin ( Φ y ( t ) ) - {A}_{y \text{-} body} ( t ) * sin ( Φ x ( t ) )}{cos ( Φ x ( t ) ) * sin ( Φ y ( t ) ) - cos ( Φ y ( t ) ) * sin ( Φ x ( t ) )}$$

$$\frac{{A}_{y \text{-} world} ( t ) = {A}_{x \text{-} body} ( t ) * cos ( Φ y ( t ) ) - {A}_{y \text{-} body} ( t ) * cos ( Φ x ( t ) )}{sin ( Φ x ( t ) ) * cos ( Φ y ( t ) ) - sin ( Φ y ( t ) ) * cos ( Φ x ( t ) )}$$

These equations are integrated once to determine horizontal and vertical velocity, and twice to determine the stride length, and the vertical displacement of the foot. While the above calculations show corrections for the pitch (X/Y) axis, it is also understood that similar corrections may be made for both roll and yaw axes as well.

### Stride Based Metrics

Referring to FIG. 3, with the full 6D device position and orientation complete, it is now possible to determine the locations of Foot Strike (A), Pronation (B), Toe Off (C), and Swing (D).

Step 1: Locate the pitch gyro peak=max(pitch gyro data) since last detected pitch gyro peak.

Step 2: Determine Foot Strike (FIG. 3-A) by searching from the previously located pitch gyro peak for the first local peak with an adaptive threshold of at least (for example) 40% of the previously detected compensated pitch gyro minimum reading (e.g. −200 deg/sec). Then looking forward to the next local minimum in the pitch gyro, noting the timestamp, pitch, roll, rate of roll (pronation rate), and yaw metrics at that location.

Step 3: Determine Toe Off (FIG. 3-C) by searching over a window of the Foot Strike detected above+10 ms to the next pitch gyro peak, whereby finding the next local trough with an adaptive threshold of at least (for example) 70% of the previously detected compensated pitch gyro minimum reading (e.g. −400 deg/sec). Again, noting timestamp, pitch, roll, and yaw metrics for this location.

Step 4: Determine Maximum Pronation Angle (FIG. 3-B) by looking between the above determined Foot Strike (FIG. 3-A) and Toe Off (FIG. 3-C) for the maximum difference between the roll noted at Foot Strike. Noting timestamp, pitch, roll, roll rate, and yaw metrics for this location. Also classifying the type of Foot Strike among (Rear Foot, Mid Foot, Fore Foot).

Steps 5-N: Continue locating all other Stride Based Metrics—including, but not limited to:
PitchMax.Pitch=max(Pitch) between Pitch Peaks [just prior to Foot Strike]
PitchMax.Roll=Roll at location of Pitch Max,
PitchMin.Pitch=min(Pitch) between Pitch Peaks [rear-most portion of Swing],
PitchMin.Roll=Roll at location of Pitch Max,
Contact.Time=Toe Off (i)−Foot Strike (i),
Cycle.Time=Foot Strike (i)−Foot Strike (i−1),
StrideRate=1/Cycle·Time,
G-Force Estimate=\sqrt{Ax²+Ay²+Az²}

The above kinematic metrics being recorded in Data Storage Memory 28 and optionally transmitted in real time via Wireless Transceiver 29 using, for example, wireless protocols such as ANT, ANT+, or Bluetooth Low Energy (BT Smart), as shown in FIG. 7.

### Real World

### Calibration, Bias Compensation, and Axial Cross-Talk

It is understood that in ideal (laboratory) environments, the sensors used to collect the kinematic parameters described above can operate with few error sources. That the data is ‘accurate’ as a result of the constrained environmental and operational settings. However, when the device is preferably used in non-laboratory settings, such as training and competition, the system must be capable of maintaining accuracy in order to continue to correctly determine the same high quality kinematic metrics as disclosed above. In order to do so, it is required that the device limitations be well understood, and compensated for accordingly.

### Limitations of Gyroscopes

The output of rate gyroscopes is rotational rate, and to obtain a relative change in angle, a single integration on the gyro outputs must be performed. Error in gyro bias (the output of the gyro when rotation is zero) leads to an error that increases with integration time. Methods must be taken to compensate for these bias errors, which are caused by drift due to time and temperature, and by noise.

### Bias Compensation of Gyroscopes

Common methods of compensation involve the use of other sensors, such as accelerometers for tilt angle, and compasses for heading. Alternately, changes in bias may be sensed when the device is not moving (i.e. pause during a run). No motion is detected by looking at peak deviation in gyro output during a relatively short timeframe, such as two seconds. If the peak-to-peak signal is below a predetermined threshold, it is determined that the device is stationary, and the average gyro output during that time becomes the new bias setting.

### Bias Compensation of Accelerometers

Note that accelerometers and compass sensors also have bias drift, but since accelerometers provide tilt angle directly (without integration) by measuring gravity, and since compass sensors provide heading information directly by measuring the earth's magnetic field, bias errors in these sensors are not integrated when providing tilt angle or heading. However, when double integrating the output of an accelerometer to provide distance or when single integrating its output to provide velocity, the bias errors of the accelerometer become important.

### Bias Compensation of Magnetic Sensors

Magnetic sensors (also known as compass sensors) are used to determine heading (yaw orientation) using magnetic north as a reference. The value of compass sensors is that they provide absolute heading information using a known reference (magnetic north). This is in contrast with gyros, which provide relative outputs that can accurately detect how far a device has rotated. Additionally, the compass sensors are typically only used for rotational information around the yaw axis, while gyros provide information around the X, Y, and Z axes (pitch, roll, and yaw).

Magnetic sensors respond to more than just the earth's magnetic field (typically ranging from 30 microteslas to over 60 microteslas). They also respond to interference, such as RF signals (caused by cell phones, radio towers, etc.) and to magnetic fields caused by magnets, such as those in cell phones and headphones. Compasses are often used in combination with gyroscopes, where the gyroscopes provide a heading signal for faster motions, and the filtered compass output provides a heading signal with a longer time constant to be used for bias and heading compensation. Additionally, since the earth's magnetic field is not perfectly parallel to the surface of the earth, its angle varies with position on the Earth, accelerometers are used in conjunction with compass sensors to provide tilt compensation.

### Roll/Yaw Axial Cross Talk

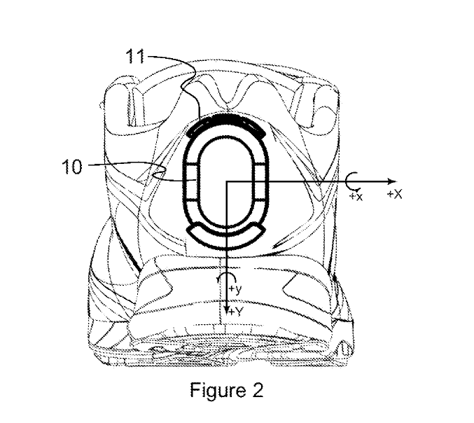

Another source of error may arise from the arbitrary mounting angle of the detachable motion sensor 10. While it is possible to vertically align the +Y axis as shown in FIG. 2, it is not always possible to horizontally align the +Z axis shown in FIG. 1. Variances in the construction of the rear of the shoe may place the device at large (e.g. 30 deg) angles from the preferable vertical orientation. In these circumstances, there will be inherent coupling between the roll and yaw gyroscope axes. Whereby a change in roll orientation of the shoe will be observed in the data for both the Z and Y axis gyroscopes (FIG. 1). One preferred method which may be used in order to correct for the cross coupling, is taken from another application, as described below.

Zero offset correction of depth is one of the first considerations in analyses of diving behaviour data from time-depth recorders (TDRs). Pressure transducers in TDRs often “drift” over time due to temperature changes and other factors, so that recorded depth deviates from actual depth over time at unpredictable rates.

For diving animals, such as marine mammals and seabirds, the problem of zero offset correction is simplified by the cyclical return to or from the surface as study animals perform their dives throughout the deployment period, thereby providing a reference for calibration (The short period where the foot is flat on the ground during each stride is the equivalent in kinematic stride analysis).

The method consists of recursively smoothing and filtering the input time series using moving quantiles. It uses a sequence of window widths and quantiles, and starts by filtering the time series using the first window width and quantile in the specified sequences. The second filter is applied to the output of the first one, using the second specified window width and quantile, and so on. In most cases, two steps are sufficient to detect the surface signal in the time series: the first to remove noise as much as possible, and the second to detect the surface level. Depth is corrected by subtracting the output of the last filter from the original.

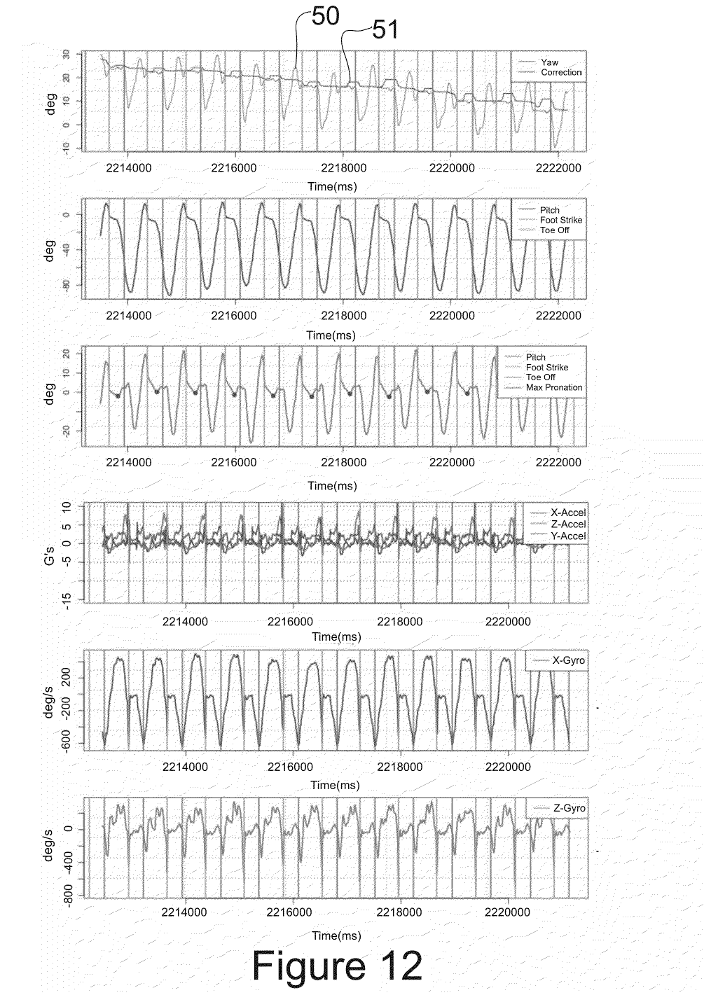

Using the above dual filter technique, the ‘corrupted’ roll and yaw data can be recursively filtered as depicted in FIG. 12. Here the Yaw Correction 51 is the result of the above described filtering method—selecting a quantile of (for example) 0.8 for the first step, and 0.05 for the second step. Further selecting a window of 100 samples (1 sec) for the first step and 20 samples (0.2 sec) for the second step. With a bounds of -180 to 180 degrees in the case of yaw. This correction may then be removed from the yaw data 50 to produce a compensated yaw reading from which metrics may now be calculated.

### Right/Left Asymmetry

Inherent in the biomechanics of humans is intrinsic asymmetry which can manifest itself in different ways which may adversely affect performance and even lead to injury. The ability of the disclosed invention to measure and record the motion of an athlete can provide valuable insight into these asymmetries when the motion sensing system 10 is affixed to both the athlete's left and right feet. Information particularly between Foot Strike (FIG. 3-A) and Toe Off (FIG. 3-C), including Pronation (FIG. 3-B), can be used to identify biomechanical differences between the right and left side stride mechanics. The knowledge of these differences being usable by people trained in the field to address the underlying conditions which are causing the asymmetry—including, but not limited to, functional limb length differences, tight tendons/ligaments, muscle soreness, and even selection of proper footwear (further described below).

When both right and left data are to be simultaneously recorded, the motion sensing system 10 on the left foot may be preferably designated as a slave device, forwarding its stride based metrics to the master device on the right foot, which will aggregate the data from the two systems, then record and/or transmit the information via wireless interface.

### Intensity Metric

Using the kinematic metrics collected by the system, it is possible to compute a metric that can be used to represent the intensity (runScore) of an activity. Specifically, using an equation of the form:

runScore=a*Pace+b*StrideRate+c*PronationExcursion+d*Maximum Pronation Velocity+e*ImpactGs+f*BrakingGs+ . . .

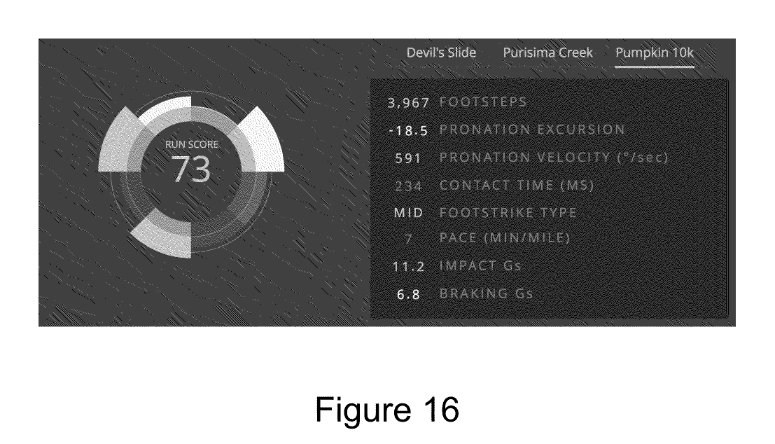

This intensity metric can then be used to quickly visualize the ‘stress’ of a given run (such as FIG. 16), enabling a user to make training decisions based on the intensity.

The intensity formula may also be expanded to further include other non-kinematic metrics, such as physiological parameters like: heart rate, heart rate variability, oxygen consumption, and perceived exertion.

### Footwear Selection

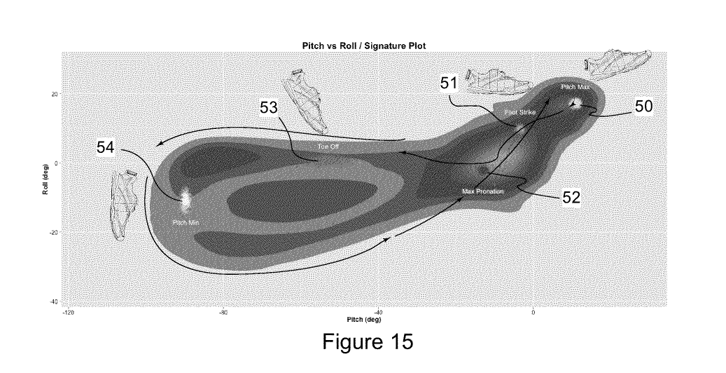

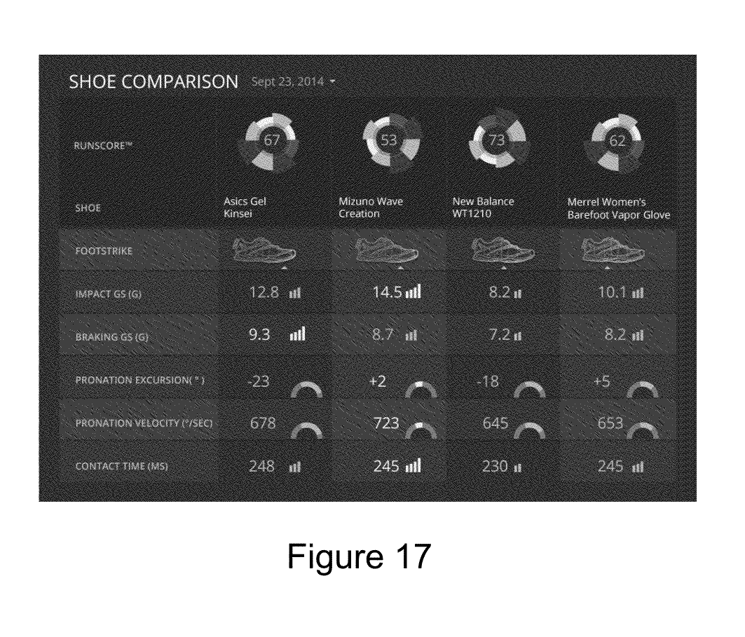

Using the data collected by the system, it is possible to interpret plots (such as FIG. 15) in order to determine the appropriate type of footwear an athlete should wear. Specifically, the area between Pitch Max 50 and Max Pronation 52 can be optimized for a specific individual by the selection of an appropriate shoe (e.g. neutral, cushion, stability/motion-control, minimalist, etc) as well as suitable orthotic devices. Furthermore, comparisons between a plurality of shoes can be made using a visualization (such as FIG. 17) to allow a user to quickly compare the individual kinematic metrics and intensity (runScore) from runs collected from each shoe.

### Shoe Wear

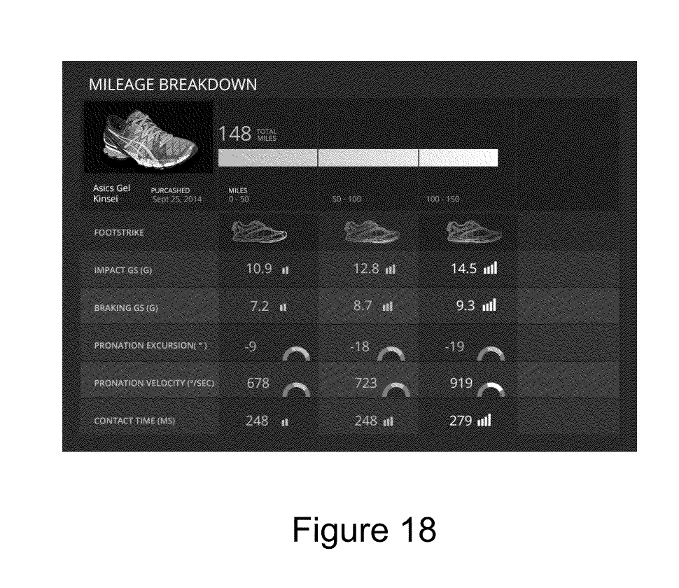

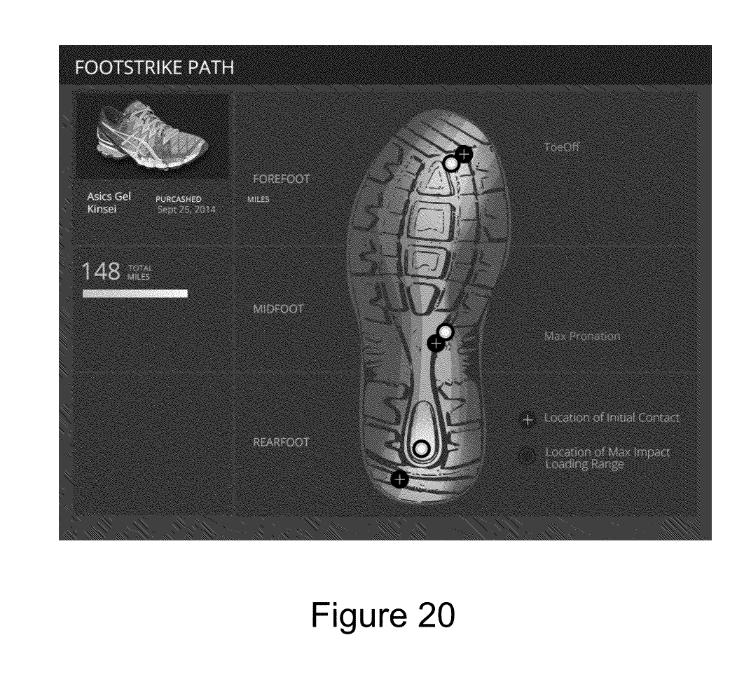

Again, using the kinematic data collected by the system, it is possible to visualize the change of kinematic parameters (such as ImpactGs, BrakingGs, Maximum Pronation Excursion) on a given pair of shoes as mileage increases (such as FIG. 18). Thus enabling a user to understand when to replace a particular pair of shoes, based on specific changes in kinematic metrics, not just based on standard mileage recommendations. Further visualizations can be made (such as FIG. 20) which show the footstrike pattern, providing a forward-look at the future wear pattern of a given pair of shoes, based on just a single use.

### Aggregate Data

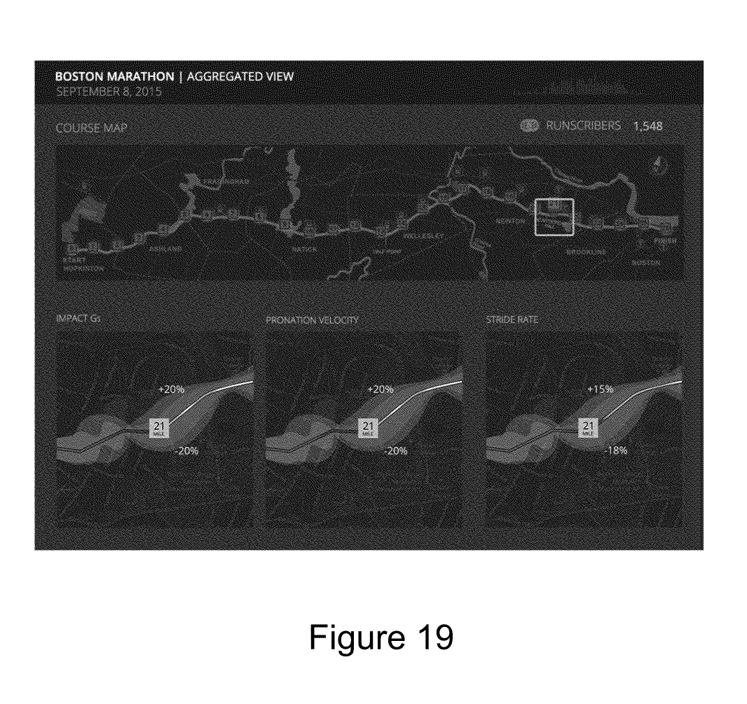

Using the data collected by the system, it is possible to aggregate kinematic metrics from a large population of users. Enabling specific demographic comparisons to be made, such as: age group, weight, competitive level, type of terrain, length of run, and average pace. Such aggregate data can then be used to enable injury correlations to the collected kinematic data, looking for trends in individual and combinations of metrics, such as ImpactGs and Pronation Velocity. The aggregate data can also be gathered for specific events which have a large number of participants (such as Boston and NYC Marathons), where the mean and variance of key kinematic metrics can be compared over the course of that specific event (shown in FIG. 19).

### Primary Components

As described above, the motion system shown in FIG. 7 includes (1) 3D Accelerometer 21, (1) 3D Gyroscope 22, and (1) 3D Compass 23, all mounted on the shoe. It is necessary that they must not interfere or influence natural gait; this requires that they are small and lightweight.

The device may be battery powered; this requires that the primary components and associated circuits possess low-power consumption characteristics.

The sensor is mounted on the foot or shoe and will thus be subjected to large impact forces and abuse. It is necessary that the sensors be rugged and durable to be able to survive in this environment.

The linearity, repeatability and noise levels must be such that the accuracy of measurement is acceptable for the application.

The motion processing units used in the development work of this invention are manufactured by InvenSense (part no.'s MPU-9150 and MPU-9250). These devices are constructed using MEMS techniques to build the transducers into a silicon chip. This accounts for the small size, low power consumption and accuracy of the devices.

The invention described herein is not limited to the above mentioned sensor family. Other MEMS accelerometers, gyroscopes, and compasses are currently produced or are under development by different manufacturers and could be considered for this purpose.

The integrated application processor and wireless transceiver used in the development work of this invention is manufactured by Nordic Semiconductor (part no.'s nRF51422, nRF51822, and nRF51922). These devices comprise an ARM Cortex-MO level microcontroller with 256 kB of embedded flash program memory and 16 kB of RAM.

The data storage memory used in the development of this invention is manufactured by Macronix (part no. MX25L25635EZNI). This device is a Serial Flash containing 256 Mbit (32 Mbyte) of non-volatile storage for data storage and retention.

Although the invention has been described with reference to various exemplary embodiments illustrated in the attached drawing figures, it is noted that equivalents may be employed and substitutions made herein without departing from the scope of the invention as recited in the claims. Having thus described embodiments of the invention, what is claimed as new and desired to be protected by patent includes the following:

### REFERENCES

### Incorporated Herein by Reference

U.S. Pat. No. 5,955,667 A
U.S. Pat. No. 6,301,964 B1
US 20100204615 A1
EP 1992389 A1
US 20070208544 A1
U.S. Pat. No. 8,529,475 B2

I claim:

1. A detachable wireless measurement device capable of determining stride kinematics in each of a plurality of strides, comprising:
a detachable receptacle which securely holds the kinematic sensor to the user's footwear;
a kinematic sensor which is comprised of a three-axis accelerometer and a three-axis gyroscope;
said kinematic sensor calculating the orientation from the accelerometer and gyroscope readings, then further determining stride kinematic metrics.

2. The device of claim 1, said kinematic sensor further comprised of a digital motion processor.

3. The device of claim 1, also including a wireless transceiver.

4. The device of claim 3, wherein said kinematic metrics are wirelessly streamed for viewing in real-time on a watch, smart phone, or computing device.

5. The device of claim 1, said kinematic sensor further including a local storage device, thus enabling the kinematic metric data to be stored locally for later download via wired or wireless interface.

6. A method of calculating a metric representing the intensity of a run (runScore) based on a set of kinematic metrics.

7. The method of claim 6, where the kinematic metrics are collected using the device of claim 1.

8. The method of claim 7, where the intensity metric is calculated from among, but not limited to, the following metrics:
pitch, roll, yaw, vertical position, horizontal position, horizontal velocity, vertical velocity, distance traveled, foot strike, foot strike classification, toe off, contact time, stride rate, stride length, rate of pronation, maximum pronation, pronation excursion, rate of plantarflexion and dorsiflexion, stance velocity, stance excursion, swing velocity, and swing excursion.

9. The method of claim 6, where the intensity metric further includes physiological metrics from among, but not limited to, the following:
heart rate, heart rate variability, oxygen consumption, and perceived exertion.

10. A method of comparing kinematic data from a number of shoes for purposes of proper footwear selection for a specific individual.

11. The method of claim 10, where the kinematic metrics are collected using the device of claim 1.

12. The method of claim 10, where an intensity metric (mnScore) is calculate for each shoe.

13. The method of claim 10, where each of a plurality of kinematic metrics are compared from among, but not limited to, the following metrics:
pitch, roll, yaw, vertical position, horizontal position, horizontal velocity, vertical velocity, distance traveled, foot strike, foot strike classification, toe off, contact time, stride rate, stride length, rate of pronation, maximum pronation, pronation excursion, rate of plantarflexion and dorsiflexion, stance velocity, stance excursion, swing velocity, and swing excursion.

14. A method of tracking shoe wear using a plurality of kinematic metrics.

15. The method of claim 14, where the kinematic metrics are collected using the device of claim 1.

16. The method of claim 14, where the intensity metric (mnScore) is calculated as the shoe's mileage increases.

17. A method of aggregating data from a large population of users for the purpose of comparing kinematic metrics.

18. The method of claim 17, where the kinematic metrics are collected using the device of claim 1.

19. The method of claim 17, where such aggregate data can be used for purposes of obtaining demographic information over specific user groups or types.

20. The method of claim 17, where such aggregate data can be used to enable an individual user to compare their personal data to demographic data obtained from the aggregate database.

21. The method of claim 17, where such aggregate data can be gathered at specific events (e.g. Boston Marathon), and used to facilitate statistical analysis of kinematic metrics over a large population of users.

22. A method for determining stride length, and thus the distance traveled, using the swing excursion of a user's footwear.

23. The method of claim 22, where the swing excursion is obtained using the device of claim 1.
